(19) United States
(12) Patent Application Publication
(10) Pub. No.: US 2026/0209827 A1
(43) Pub. Date: Jul. 23, 2026

(54) STERILISED APPARATUS FOR CAPTURING NUCLEIC ACIDS FROM BODY FLUIDS’

(71) Applicant: BIOCAPTIVA LIMITED, Scotland (GB)

(72) Inventor: Matthew John Simmonte Owens, Scotland (GB)

(73) Assignee: BIOCAPTIVA LIMITED, Scotland (GB)

(21) Appl. No.: 19/142,136

(22) PCT Filed: Dec. 21, 2023
(86) PCT No.: PCT/GB2023/053351

§ 371 Date: Jun. 20, 2025

(30) Foreign Application Priority Data
Dec. 21, 2022 (GB) 2219468.2
Dec. 21, 2022 (GB) 2219472.4

(51) Int. Cl. C12Q 1/6806

(52) U.S. Cl. CPC C12Q 1/6806

(57) ABSTRACT

The present invention relates to a method of preparing an apparatus configured to capture a nucleic acid from a sample, comprising providing a substrate, wherein the substrate is a nucleobase-containing polymer or is provided with said polymer, and treating the substrate and/or the polymer with ethylene oxide. The invention further relates to the apparatus so prepared, methods and uses thereof.

### FIELD OF THE INVENTION

The present invention is in the technical field of preparing an apparatus configured to capture a nucleic acid from a sample, particularly preparing a sterilised apparatus configured to capture cell free DNA (cfDNA) from a bodily content (e.g. bodily fluid). The present invention is also in the technical fields of the apparatus per se, methods and uses thereof.

### BACKGROUND OF THE INVENTION

Liquid biopsy is a non-invasive, rapid and precise technique that has made big impact in the field of cancer treatment. It can be derived from blood, urine, plasma or other bodily fluids that include biomarkers. A typical example of the biomarker is circulating tumour DNA (ctDNA) which is a component of a larger cfDNA freely circulating in the bloodstream. Liquid biopsy can be used to sample and analyse cfDNA for the presence of ctDNA markers. Said ctDNA markers may be present even before a tumour is big enough to be visible on a scan.

A bottleneck of the liquid biopsy is that the ratio of cancerous biomarkers (e.g. cfDNA) to non-cancerous biomarkers is usually very low at early and residual stages of the cancer management. In other words, cfDNA is usually present in an extremely small concentration. This problem is further acerbated when the sample source is limited to common medical practice such as a phlebotomy, which only offers about 10 mL venous blood. Insufficient quantity of cfDNA may make cancer detection difficult. To mitigate the problem, other techniques such as DNA amplification have been used in conjunction with liquid biopsy. However, this way forward has its own drawbacks. For example, the operation can be cumbersome, and it increases the chance of diagnostic errors. The DNA amplification may have variable reaction fidelities, resulting in amplification errors and increasing the chance of diagnostic errors.

Under this context, it is desirable to employ highly efficient apparatus for capturing cancerous biomarkers (e.g. cfDNAs), such that the total quantity of input can be sufficient for liquid biopsy diagnostics. Preferably, the apparatus also releases the captured markers in a recovery process for further, separate analyses.

To our knowledge, there have been developments of materials and devices that offer certain affinity to nucleic acids, for example, cfDNAs.

WO 2019/053243A1 describes a device configured to perform apheresis comprising one or more affinity matrices, wherein said one or more affinity matrices are capable of capturing nucleosome-bound cell free DNA (cfDNA), exosome-bound cfDNA, and unbound cfDNA from blood or plasma of a subject.

‘Synthesis and characterization of nucleobase-containing polyelectrolytes for gene-delivery’ by Eveline Maria Van Der Aa, a thesis submitted to Virginia Polytechnic Institute and State University for a degree of Master, 5 May 2010, pages 1-84 describes gene delivery with adenine- and thymine-containing poly dimethylamino ethyl methacrylate. Said polymer is used to bind plasmid DNAs.

WO 2019/097232A1 describes methods for isolating nucleic acids present in a sample, in particular cell-free DNA (cfDNA) from a blood sample and polymers, substrates and kits for the method. Polymers with characteristics suitable to bind such nucleic acids are also provided.

Despite the prior art, there is a continued need to prepare highly efficient apparatus for capturing as well as recovering nucleic acids (e.g., cfDNA). The apparatus so prepared may be sterilised for medical use and it is ideal that the sterilised apparatus at least maintains its efficacy of capturing and recovering biomarkers (e.g., cfDNAs). Thus, it is particularly beneficial to prepare a sterilised apparatus that offers improved efficacy. The present invention has been devised in this context.

### SUMMARY OF THE INVENTION

In a first aspect of the invention, there is provided a method of preparing an apparatus configured to capture a nucleic acid from a sample, comprising the steps in sequence of: (i) providing a substrate, wherein the substrate is a nucleobase-containing polymer or is provided with said polymer; and (ii) treating the substrate and/or the polymer with ethylene oxide. The nucleic acid is preferably a cfDNA. The sample can be blood (e.g. whole blood) or plasma, preferably plasma. The apparatus may also be configured to recover a nucleic acid from a sample. In other words, the apparatus can release at least part of (i.e. some of) and preferably all of the captured nucleic acid for subsequent liquid biopsy analyses. It may be that the apparatus is configured to reversibly capture the nucleic acid. It will be understood that the substrate may refer to the substrate that is the nucleobase-containing polymer, and/or the (original) substrate without provision of the polymer, whichever is appropriate under the context of the present invention.

Herein, the substrate may be formed of the nucleobase-containing polymer. Alternatively, the substrate may be formed of a different material (e.g. polyurethane) and said substrate may be provided with the nucleobase-containing polymer. For instance, said substrate may be coated by the nucleobase-containing polymer. In a second aspect of the invention, there is provided an apparatus prepared according to the method of the first aspect of the invention. Preferably, the apparatus is contained within a packaging. Preferably, the packaging is a primary packaging such as pouches.

In a third aspect of the invention, there is provided a method for preserving or improving capture of a nucleic acid from a sample, comprising the steps of: (i) providing a sample comprising a nucleic acid; (ii) providing an apparatus according to the second aspect; (iii) providing the sample of step (i) to the polymer comprised in the apparatus of step (ii), and (iv) capturing the nucleic acid by the polymer.

In a fourth aspect of the invention, there is provided use of ethylene oxide to preserve or improve capture of a nucleic acid from a sample, wherein ethylene oxide is used to treat a substrate and/or a polymer. The substrate and the polymer are comprised in an apparatus. The treated apparatus is used to capture a nucleic acid.

The method and use may also be for recovering nucleic acid from a sample. Herein, it will be understood that the word ‘recovery’, ‘recovering’ or ‘recovered’ refers to releasing at least part of the captured nucleic acids (preferably releasing all captured nucleic acids), meaning that the captured acids are at least partially (preferably fully) separated and/or removed from the apparatus. An additional step of releasing at least some of the captured acid from the apparatus may be included. It may be that the captured acid is partially or fully released from the apparatus. It may be that the captured acid is released partially or fully from the nucleobase-containing polymer. Optionally, the released nucleic acid is purified. Optionally, the method and use comprise purifying the released nucleic acid.

Herein, ‘preserving’ refers to maintaining the efficacy of the apparatus. For example, the apparatus can at least capture and/or recover the same level of nucleic acid as prior to ethylene oxide treatment. In other words, the efficacy of the apparatus is not compromised by the ethylene oxide treatment. By ‘improving’ is meant the apparatus can provide increased level of nucleic acid in comparison to an apparatus without ethylene oxide treatment. The apparatus can provide increased nucleic acid capture and/or recovery.

In a fifth aspect of the invention, there is provided use of ethylene oxide to manufacture an apparatus.

In the fourth and the fifth aspects, the substrate is a nucleobase-containing polymer or is provided with said polymer. Preferably in these aspects, the substrate, the polymer, and the treatment with ethylene oxide are as described in the first aspect of the invention.

These and other aspects, features and advantages will become apparent to those of ordinary skill in the art from a reading of the following detailed description and the appended claims.

### DETAILED DESCRIPTION OF THE INVENTION

### Nucleic Acids and the Sample

In the context of the present invention, the sample is typically a bodily content (e.g. a bodily fluid), preferably a blood or a serum or a plasma, more preferably a plasma. The volume of the sample is typically from 1 mL to 100 mL, more typically from 5 mL to 50 mL, still more typically from 10 mL to 25 mL. In a preferred example, the sample (e.g. blood or plasma) is 10 mL. The above-described volumes are typical for a fluid collector (e.g. the apparatus that comprises (e.g. is) a fluid collector). It is also possible that the sample volume is from 200 mL to 5 L, or from 500 mL to 3 L, or from 500 mL to 1 L, which is typical for an apheresis procedure (e.g. for an apheresis apparatus). It may be that the sample volume is from 20 mL to 2 L, or from 30 mL to 1 L, or from 50 mL to 500 mL.

The nucleic acid comprised in the sample is preferably a cfDNA, more preferably a ctDNA. Examples of cfDNAs include but are not limited to nucleosome-bound cfDNAS, exosome-bound cfDNAs, unbound cfDNAs and mixtures thereof. It may be that the nucleic acids are selected from single-stranded DNAs, single-stranded RNAs, double-stranded DNAs, double-stranded RNAs, and mixtures thereof.

Nucleic acids (e.g. cfDNAs) may also be selected from double-stranded DNAs, single-stranded DNAs, oligonucleotides, and mixtures thereof. A single-stranded nucleic acid, such as a single-stranded DNA (e.g. ss cfDNA), may comprise from 20 bases to 500 bases, typically from 50 bases to 400 bases, more typically from 100 bases to 300 bases. It is also possible for a single-stranded nucleic acid such as a single-stranded DNA (e.g. ss cfDNA) to comprise large fragments, for example, from 1 kilobases (kb) to 1000 kb, typically from 10 kb to 800 kb, more typically from 50 kb to 700 kb, still more typically from 100 kb to 500 kb. A suitable example of a single-stranded DNA (e.g. ss cfDNA) comprises 160 bases. A double-stranded nucleic acid, such as a double-stranded DNA (e.g. ds cfDNA), may comprise from 50 base pair (bp) to 500 bp, typically from 100 bp to 400 bp, more typically from 200 bp to 300 bp. It is also possible for a double-stranded nucleic acid such as a double-stranded DNA (e.g. ds cfDNA) to comprise large fragments, for example, from 1 kilo base pair (kbp) to 1000 kbp, typically from 10 kbp to 800 kbp, more typically from 50 kbp to 700 kbp, still more typically from 100 kbp to 500 kbp. A suitable example of a double-stranded DNA (e.g. ds cfDNA) comprises 160 bp.

Typically, the concentration of the nucleic acid (e.g. cfDNA) in the sample is from 0.1 ng/mL to 100 ng/mL, more typically from 1 ng/ml to 100 ng/ml, still more typically from 2 ng/ml to 70 ng/ml, most typically from 5 ng/ml to 50 ng/ml. In a suitable example, the concentration of the nucleic acid (e.g. cfDNA) is 10 ng/ml. Herein, the concentrations refer to the original nucleic acid concentrations in the samples (prior to any capture by the apparatus). Typically, the apparatus can capture 10% to 90% nucleic acid comprised in the sample (e.g. for a 10 ml sample comprising 100 ng nucleic acid in total, the apparatus can capture from 10 ng to 90 ng nucleic acid.), more typically from 30% to 85%, still more typically from 50% to 80%. Typically, the density of the captured nucleic acid on the substrate is from 0.1 ng/cm³ to 100 ng/cm³, more typically from 0.5 ng/cm³ to 50 ng/cm³, still more typically from 1 ng/cm³ to 20 ng/cm³, most typically from 3 ng/cm³ to 10 ng/cm³. It may be that the density is at least 5 ng/cm³, or at least 20 ng/cm³, or at least 30 ng/cm³, or at least 50 ng/cm³, or at least 60 ng/cm³. Optionally, it may be that the density is no more than 100 ng/cm³, or no more than 80 ng/cm³, or no more than 70 ng/cm³. Typically, the nucleic acid recovery per 1 cm² surface area of the substrate is from 0.1 ng to 50 ng, or from 0.2 ng to 30 ng, or from 1 ng to 20 ng, or from 1 ng to 10 ng, or from 1 ng to 5 ng. It may be that the nucleic acid recovery per 1 cm² surface area of the substrate is at least 5 ng, or at least 20 ng, or at least 30 ng, or at least 50 ng, or at least 60 ng. Optionally, it may be that the nucleic acid recovery per 1 cm² surface area of the substate is no more than 100 ng, or no more than 80 ng, or no more than 70 ng. Typically, the recovery rate of the apparatus is from 5% to 90% (e.g. for a 10 ml sample comprising 100 ng nucleic acid in total, the apparatus can recover 5 ng to 90 ng nucleic acid), more typically from 10% to 80%, still more typically from 15% to 70%. Typically, the apparatus can recover from 1 ng to 5000 ng nucleic acid, or from 1 ng to 2000 ng nucleic acid, or from 5 ng to 1000 ng nucleic acid, or from ng to 950 ng, or from 100 ng to 900 ng, or from 500 ng to 900 ng. The above-described capture and recovery of nucleic acid can be achieved by a single or double uses of the apparatus.

The apparatus so prepared according to the present invention may be configured to capture nucleic acids from the sample as described herein. The apparatus may be configured to use one or more times (i.e. single use or multiple uses) to capture nucleic acids from the same or different samples.

### Preparation of the Apparatus

It will be understood that the preparation of an apparatus comprising the steps of providing a substrate wherein the substrate is a nucleobase-containing polymer or is provided with said polymer, and treating the substrate and/or the polymer with ethylene oxide.

The polymer can efficiently capture (e.g. bind) the nucleic acid (e.g. cfDNA) from the sample (e.g. plasma), preferably without capturing other components from the sample. Said polymer may also efficiently release the captured nucleic acid for the practitioners to recover adequate amount of nucleic acid for subsequent analysis.

The substrate may be a matrix, typically a porous matrix. The pore size of the substrate may be from 50 μm to 2000 μm, typically from 100 μm to 1500 μm, more typically from 200 μm to 1000 μm. The pore density (pore per inch/ppi) of the substrate may be from 15 ppi to 100 ppi, or from 20 ppi to 95 ppi, or from 40 ppi to 90 ppi, or from 50 ppi to 90 ppi. The substrate may comprise a plurality of openings. For example, the substrate May be a mesh or a sponge. Preferably, the substrate is a sponge. Suitably, the substrate may be in the form of a plurality of beads. Herein, beads refer to particles that are spherical or irregular in nature. The size of beads optionally ranges from 0.1 μm to 1 mm in diameter, or from 1 μm to 100 μm, or from 2 μm to 50 μm, or from 5 μm to 10 μm. Typically, the volume of the substrate is from 1 cm³ to 150 cm³, more typically from 2 cm³ to 100 cm³, still more typically from 3 cm³ to 90 cm³, most typically from 5 cm³ to 90 cm³ (e.g. 80 cm³). It may be beneficial for the substrate to have the volume as described herein since the substrate as well as the resultant apparatus may be easy to handle (e.g. portable), easy to process and/or easy to prepare. In other words, a small and compact substrate (e.g. a small and compact apparatus) may be used to achieve the desired efficacy. It may be that the volume refers to volume of the substrate which is not the nucleobase-containing polymer and without provision of said polymer (i.e. the original, naked substrate). It may be that the volume refers to the volume of the substrate which is the nucleobase-containing polymer.

Typically, the substrate permits liquid (e.g. blood) to pass through. Typically, the substrate permits non-nucleic acid molecules from the liquid (e.g. blood) to pass through while capturing nucleic acid molecules. The liquid is typically an aqueous composition comprising nucleic acids. Typically, the substrate permits release of the captured nucleic acid molecules for subsequent analysis. Typically, the substrate comprises a surface, and preferably the nucleobase-containing polymer is provided on said surface. During use, the nucleic acid from the sample can bind onto said surface.

In embodiments wherein the substrate is provided with the nucleobase-containing polymer, the polymer may be provided between and/or on the plurality of openings of the substrate (e.g., sponge). For example, the polymer may be coated on the substrate (e.g., sponge), and/or coated on openings of the substrate (e.g. sponge). If the substrate comprises a surface, the polymer may be provided (e.g. coated) on said surface. The polymer may be provided (e.g. coated) between the openings of the substrate (i.e., the polymer does not cover the openings of the substrate). It may be that the polymer is coated on the substrate (e.g. sponge). It may be that some of the openings of the substrate (e.g. sponge) are covered by the polymer. It may be that some of the solid constituents of the substrate (e.g. sponge) are covered by the polymer. It may be that (some of) the openings of the substrate (e.g. sponge) are not covered by the polymer. Typically, the polymer covers not more than 50% of the openings (with respect to the total number of openings) of the substate, or not more than 20%, or not more than 10%, or not more than 5%, or not more than 1%. In these and other embodiments, the substrate may comprise (e.g. be) polyurethane. Preferably, the substrate is a polyurethane sponge.

Preferably, the specific surface area of the substrate is at least 10 cm²/cm³. Suitably, the specific surface area is at least 15 cm²/cm³, or at least 20 cm²/cm³, or at least 25 cm²/cm³, or at least 35 cm²/cm³. But optionally, the specific surface area may be not more than 60 cm²/cm³, or not more than 50 cm²/cm³, or not more than 45 cm²/cm³. The specific surface area may be from 20 cm²/cm³ to 50 cm²/cm³, or from 35 cm²/cm³ to 45 cm²/cm³. It may be that at least part of the substrate or preferably the whole substrate has the specific surface area as described herein. It may be that the specific surface area (SSA) refers to SSA of the substrate which is not the nucleobase-containing polymer and without provision of said polymer (i.e. the original, naked substrate). It may be that the specific surface area refers to SSA of the substrate which is the nucleobase-containing polymer or coated with the nucleobase-containing polymer.

The specific surface area can be determined in accordance with established methods, for example, ASTM F2450-18. Nitrogen absorption-based isotherms (NAI) or mercury intrusion porosimetry (MIP) may be used.

It may be that the specific surface area is measured and calculated as set out below. A section of the substrate is imaged under magnification in reference to a known, calibrating distance, using a calibration microscope slide or graticule. The acquired digital image is uploaded into a suitable image analysis software (e.g. ImageJ/FIJI) and the calibrating distance is used to set the correct scale. After this, the diameter of the substrate filaments (e.g. solid polyurethane struts formed around the pores and openings) can be measured and a mean diameter (d) or radius (r) can be calculated. The specific surface area (cm²/cm³) can be calculated for a given volume of substrate provided the density of the material (e.g. polyurethane) and the mass of the required volume are known.

$$mass of substrate / density of polyurethane = volume of polyurethane$$

The filaments of the substrate have been observed to be cylindrical, thus it can be assumed that the entire volume of the material (e.g. polyurethane) follows the calculation for the volume of a cylinder and the subsequent rearrangement:

$$volume of polyurethane = π {r}^{2} \times L$$

$$L = volume of polyurethane / π {r}^{2}$$

With a known length (L) of cylindrical filament equating to all the material (e.g. polyurethane) within a given substrate volume, the surface area of a hypothetical single, long cylinder can be calculated, providing the total surface area of the material (e.g. polyurethane) within the substrate:

$$surface area of filament = 2 π rL + 2 π {r}^{2}$$

$$specific surface area = \frac{surface area of filament}{volume of polyurethane}$$

The method specified herein can be referred to as an ‘Imaging Method’. It will be understood that other methods of measuring specific surface area are widely known and may be suitable. The methods (e.g. ‘Imaging Method’) may be applicable to materials in general including polyurethane materials. The methods (e.g. ‘Imaging Method’) may also be used to determine the volume and/or the total surface area of the substrate.

It is found that the increase of specific surface area leads to further improvement of efficiency with respect to nucleic acid capture and/or recovery. The identified ranges as described herein are particularly preferred. Especially, the substrates with identified ranges offer dramatic increase of nucleic acid recovery from plasma samples (e.g. a seven-fold increase of recovery from a plasma, when the specific surface area increases only two folds). Further, without wishing to be bound by any theory, it is believed that if the specific surface area is too high (i.e., the substrate is too dense), it is possible that part of the sample will not travel through but merely travel around the substrate. In other words, the capture and/or recovery of nucleic acid may reach a maximum (and possibly plateau with further increase of specific surface area). Overall, if the specific surface area is equal to or higher than the identified lower limit (e.g. at least 10 cm²/cm³), the apparatus may provide the benefits (e.g., improved DNA recoveries).

The total surface area of the substrate may be at least 20 cm², or at least 30 cm², or at least 40 cm², or at least 100 cm², or at least 500 cm², or at least 800 cm², or at least 1500 cm². The surface area of the substrate may optionally be no more than 8000 cm², or no more than 6000 cm², or no more than 5000 cm², or no more than 4000 cm², or no more than 3600 cm². The identified total surface area may optimise the efficacy of the nucleic acid recovery. The total surface area may be defined by the volume and the specific surface area (e.g. see above, the ‘imaging method’ wherein the total surface area=volume×specific surface area). The total surface area may refer to the total surface area of the substrate which is not the nucleobase-containing polymer and without provision of the polymer (i.e. the original, naked substrate). The total surface area may refer to the total surface area of the substrate which is the nucleobase-containing polymer.

The shape of the substrates may be designed by those skilled in the art. The substrates may be polyhedrons or non-polyhedrons. It may be that the substrate is in cylindrical or substantially cylindrical shape. It may be that the substrate is in cubic or substantially cubic shape. For example, the substrate may be in cubic shape. The cubic or substantially cubic shape may optimise the efficacy of nucleic acid recovery. A polyhedron or non-polyhedron (e.g. preferably polyhedron) substrate may have the specific surface area as described herein, and/or the volume as described herein, and/or the total surface area as described herein.

It may be that the substrate has the specific surface area as described herein, and/or the volume as described herein, and/or the total surface area as described herein. It may be that the substrate has a specific surface area of at least 10 cm²/cm³ (e.g. from 20 cm²/cm³ to 50 cm²/cm³) and a volume of at least 2 cm³ (e.g from 5 cm³ to 90 cm³). It may be that the substrate has a specific surface area of at least 10 cm²/cm³ (e.g. from 20 cm²/cm³ to 50 cm²/cm³) and a total surface area of at least 20 cm² (e.g. from 1500 cm² to 5000 cm²). It may be that the substrate has a volume of at least 2 cm³ (e.g from 5 cm³ to 90 cm³), and a total surface area of at least 20 cm² (e.g. from 1500 cm² to 5000 cm²). It may be that the substrate has a specific surface area of at least 10 cm²/cm³ (e.g. from 20 cm²/cm³ to 50 cm²/cm³), a volume of at least 2 cm³ (e.g from 5 cm³ to 90 cm³), and a total surface area of at least 30 cm² (e.g. from 1500 cm² to 5000 cm²).

Typically, the substrate comprises polyurethane. Typically, the substrate is formed from polyurethane. The main types of polyurethane are polyether urethane and polyester urethane. Polyester urethane is preferred. Particular benefits of polyurethane may include enhanced flexibility, strength, durability and comfort (e.g. if the apparatus is at least partly intracorporeal). Other materials such as PVC may also be used but they may not provide all the benefits (e.g. PVC substrate may be brittle and cannot be used multiple times). Typically, the polyurethane is formed by reacting a di- or triisocyanate with a polyol. The polyurethane may comprise polyol(s), di- or triisocyanate(s) and chain extender(s). The chain extender may typically comprise one or more hydroxyls or amine groups (e.g., water, diol or diamine). Di- or triisocyanates may be aromatic or aliphatic, preferably aliphatic. The polyurethane used may be a linear, branched, or crosslinked polymer. The polyurethane may be in the form of elastomer, film, membrane, hydrocolloids, fibre, foam, or mixtures thereof. The polyurethane may be reticulated or unreticulated (preferably reticulated). The substrate may comprise polyurethane which optionally comprises one or more additives, such as plasticisers, catalysts, foaming agents, surfactants, pigments, fillers, flame retardants, antibacterial agents, antifungal agents, and mixtures thereof. Preferably, the substrate comprises (e.g. is) a polyurethane sponge. Preferably, the polyurethane sponge has the pore size as described above. The pores may be open or closed pores, preferably open pores. By ‘open’ is meant the pore is not completely closed such that a liquid (e.g. blood) can pass through. An open pore may provide pathway to at least one of the other pores and/or to other parts of the substrate. The substrate (e.g. polyurethane sponge) may comprise at least 50% open pores with respect to its total pore population on number basis, or at least 60% open pores, or at least 80% open pores, or at least 90%, or at least 95%, or 100%. The polyurethane sponge density may be from 20 kg/m³ to 1200 kg/m³, or from 50 kg/m³ to 1000 kg/m³, or from 90 kg/m³ to 800 kg/m³. It may be that the polyurethane sponge density is at least 10 kg/m³, or at least 20 kg/m³, or at least 30 kg/m³. Optionally, it may be that the polyurethane sponge density is no more than 100 kg/m³, or no more than 50 kg/m³, or no more than 40 kg/m³. The stiffness of the polyurethane sponge may be from 20 A to 80 D, or from 40 A to 40 D, or from 50 A to 10 D, in accordance with the Shore Hardness Scale. It will be understood that the substrate comprising polyurethane (e.g. a polyurethane substrate) may have the specific surface area, and/or the volume, and/or the total surface area, as described herein. It may be that said substrate has the shape as described herein.

When the substrate is provided with a nucleobase-containing polymer, the polymer may be chemically and/or physically attached to the substrate. The chemical attachment may be via one or more chemical interactions. Said interactions may be covalent, and/or ionic, and/or via electrostatic interactions, and/or hydrogen bonding. The physical attachment may be a physical adsorption and/or hydrophobic effect or entrapment. The polymer may be adsorbed onto the substrate. The polymer may be coated on the substrate. The polymer may be provided on the substrate (e.g. coated on the substrate) in the form of one or more layers. The layered morphology of the polymer may lead to improved nucleic acid capture and/or recovery.

It may be that at least 70% of the surface area of the substrate is provided with (e.g. coated with) the polymer. In other words, the polymer covers at least 70% of the surface area of the substrate. Alternatively, the polymer covers at least 80%, or at least 90% or at least 95% or 100% of the surface area of the substrate. The weight ratio of the polymer to the substrate may be from 1/1000 to 1/1, or from 1/500 to 1/20, or from 1/100 to 1/50. The density of the polymer on the substrate may be from 0.001 g polymer per 1 cm³ substrate (g/cm³) to 1 g/cm³, or from 0.01 g/cm³ to 0.5 g/cm³, or from 0.05 g/cm³ to 0.1 g/cm³. It may be that the density of the polymer on the substrate is at least 0.00001 g/cm³, or at least 0.0001 g/cm³, or at least 0.0002 g/cm³, or at least 0.0003 g/cm³. Optionally, it may be that the density is no more than 0.01 g/cm³, or no more than 0.001 g/cm³, or no more than 0.0008 g/cm³. The polymers may be anchored to the substrate. Additionally and optionally, at least part of the polymers may penetrate the substrate. The depth of penetration may be at least 0.1% of the total depth of the substrate, or at least 1%, or at least 2%, or at least 5%, or at least 10%, or at least 20%, but not more than 50%, or not more than 40% or not more than 30%.

When the substrate is provided with the nucleobase-containing polymer, it may be that the provision of the polymer does not alter (e.g. substantially alter) the specific surface area of the substrate, and/or the volume of the substrate, and/or the total surface area of the substrate, and/or the shape of the substrate. In other words, the substrate provided with the polymer may have substantially the same (e.g. the same) volume, and/or specific surface area, and/or total surface area, and/or shape as the original substrate.

The polymer morphology and/or the polymer coverage described herein may be determined by any known and effective methods. For example, the substrate provided with the polymer may be viewed by scanning electron microscopy (SEM) or any suitable microscopy techniques. Images (e.g. in-depth images) may be obtained, and topographical analysis and/or calculations may be performed to analytically determine the polymer coverage.

### The Nucleobase-Containing Polymer

In the context of the present invention, the substrate is a nucleobase-containing polymer or provided with said polymer. Typically, the nucleobase-containing polymer comprises a backbone with at least a proportion of side chains being nucleobase side chains, and the polymer comprises a positively charged moiety at physiological pH adapted to bind the nucleic acid via electrostatic interaction, and/or the polymer is adapted to bind the nucleic acid via hydrogen bonding. Herein, physiological pH normally refers to a pH of from 2 to 11, typically from 4 to 9, more typically from 5 to 7. In the context of the present invention, it will be understood that the polymer may be a positively charged polymer at physiological pH. ‘Positively charged’ should be understood to mean that the overall charge is positive. It is possible that the polymer is free from negatively charged moieties, typically at physiological pH. It will be further understood that the substrate of the present invention may be positively charged, typically at physiological pH. It is because the substrate is either the polymer or is provided with said polymer.

Typically, each of the nucleobase side chains comprises one or more nucleobases, independently selected from natural nucleobases, unnatural nucleobases, modified nucleobases wherein the modification allows interaction with nucleic acid via hydrogen bonding and/or electrostatic binding, and mixtures thereof. Suitable examples of the nucleobases include but are not limited to purine nucleobases, pyrimidine nucleobases, and mixtures thereof, wherein the nucleobases are optionally modified to allow the interaction as described above. The modified purine nucleobase may be based on a modified adenosine or guanosine structure. The modified pyrimidine nucleobase may be based on a modified cytosine, thymine or uridine structure.

Typically, each of the nucleobase side chains comprises one or more nucleobases, independently selected from adenine (A), cytosine (C), guanine (G), thymine (T), uracil (U), derivatives of ACGTU thereof and mixtures thereof. Derivatives of ACGTU include but are not limited to aminoadenine, aminocytosine, aminoguanine, aminothymine, aminouracil, purine, pyrimidine, hypoxanthine, xanthine, theophylline, theobromine, caffeine, uric acid, isoguanine, 7-methylguanine, 5,6, dihydrouracil, 5-methyl cytosine, 5-hydroxymethylcytosine, 3-Nitropyrrole, 5-Nitroindole, 2,6-diaminopurine, 6, 8 diaminopurine, pyrene, fluorouracil, barbituric acid, orotic acid, a salt or ester of ACGTU, a compound that releases one or more ACGTUs during use, a compound that comprises one or more ACGTUs, and mixtures thereof. Preferably, the nucleobase side chains comprise cytosine or thymine or a combination thereof. More preferably, the nucleobase side chains comprise thymine.

Typically, the molar proportion of side chains of the polymer comprising one or more nucleobases may be at least 1%, or at least 5%, or at least 10%, but not more than 20%, or not more than 15%. In preferred embodiments, each of the nucleobase side chains comprises one or more nucleobases independently selected from ACGTU, derivatives of ACGTU thereof and mixtures thereof, and the molar proportion of total said side chains of the polymer is from 1% to 20%, or from 2% to 15%, or from 5% to 15%.

Without wishing to be bound by any theory, it is believed that the nucleobase enables base-pairing of the polymer to a nucleic acid. Thus, the polymer can capture (e.g. bind) the nucleic acid from a sample. A nucleobase may allow base pairing via hydrogen bonding typically with additional electrostatic interaction, for example double or triple hydrogen bonding between amine and carbonyl groups provided on the nucleic acid and the polymer. The electrostatic interactions may be provided via tertiary ammonium ions on the polymer and phosphate esters on the nucleic acid.

Preferably, the nucleic acid is reversibly captured by the polymer via hydrogen bonding and/or electrostatic interaction. In other words, the acid may be released (e.g. in a recovery process) for further analysis when needed.

Additionally and optionally, the side chains of the polymer backbone comprise one or more of the followings: amines, amides, alcohols, carboxylic acids, alkanes, alkenes, alkynes, esters, ethers, epoxies, sulfonyl hydrides, sulfonyls, thiols, heterocycles, homocycles, aromatic cycles, anti-aromatic cycles, or derivatives thereof, or combinations thereof.

The polymer of the present invention may comprise one or more positively charged moieties at physiological pH that can bind the nucleic acid via electrostatic interaction. The positively charged moieties may attract negatively charged groups (e.g. phosphate) on nucleic acid. Typically, the positively charged moieties at physiological pH may be quaternary ammonium salts or quaternary phosphonium salts, preferably quaternary ammonium salts. Typically, the polymer comprises one or more amines (e.g. diethyl amine) that can provide a positive charge at physiological pH, more typically one or more amine side chains (e.g. diethyl amine side chains). Typically, the polymer comprises at least 5% amine side chains (e.g., diethyl amine side chains) or at least 10%, or at least 20%, or at least 30%, but not more than 50%, or not more than 40%, in which the percentages refer to molar percentages with respect to the whole polymer. In the preferred embodiments described above wherein each of the nucleobase side chains comprises one or more nucleobases independently selected from ACGTU, derivatives of ACGTU thereof and mixtures thereof, and the molar proportion of total said side chains of the polymer is from 1% to 20%, or from 2% to 15%, or from 5% to 15%, and the amine side chains (e.g. diethylamine side chains) is from 5% to 50% or from 30% to 50%.

Typically, the polymer comprises nucleobase-containing monomers and non-nucleobase monomers. Typically, the polymer is a copolymer, or a terpolymer, or a tetrapolymer, or a pentapolymer. Preferably the polymer is a tetrapolymer or a pentapolymer, more preferably a pentapolymer. The non-nucleobase monomers May be selected from 2-methoxyethyl acrylate (MEA), 2-methoxyethyl methacrylate (MEMA), diethylamino ethyl acrylate (DEAEA), diethylamino ethyl methacrylate (DEAEMA), hydroxyethyl acrylate, hydroxyethyl methacrylate, methyl methacrylate, methyl acrylate, styrene, methyl styrene, glycidyl acrylate, glycidyl methacrylate, N-vinylacetamide, 2-methyl-2-nitropropyl methacrylate, acrylic acid, methacrylic acid, 2-[[(butylamino) carbonyl]oxy]ethylacrylate, dimethylamino ethyl acrylate, dimethylamino ethyl methacrylate, mono-2-(acryloyloxy)ethyl succinate, poly(ethylene glycol) methyl ether acrylate (PEGA), poly(ethylene glycol) methyl ether methacrylate (PEGMA), and mixtures thereof, preferably selected from (meth)acrylate-based monomers thereof and mixtures thereof, more preferably selected from MEA, MEMA, DEAEA, DEAEMA, PEGA, PEGMA and mixtures thereof, most preferably selected from MEA, MEMA, DEAEA, PEGA and mixtures thereof. Typically, the polymer of the present invention comprises the monomers selected above and (meth)acrylate-based monomer with nucleobase side chains. The (meth)acrylate-based monomers with nucleobase side chains may be selected from thymine ethyl acrylate (ThEA), Thymine acetoxyethyl methacrylate (ThAcMA) and mixtures thereof. The selection of MEA, MEMA, PEGMA, PEGA and mixtures thereof as non-nucleobase monomers is particularly preferred since they provide good resistance to biofouling. The selection of PEGA, PEGMA and mixtures thereof is more preferred since they offer improved haemocompatibility by reducing coagulation in the blood. The (meth)acrylate-based monomers with poly(ethylene glycol) side chains (e.g. PEGA, PEGMA) used in the present invention may be of the same or different molecular weight.

Typically, the polymer is formed from (meth)acrylate-based monomers. Typically, the polymer of the present invention comprises (meth) acylate-based monomers with nucleobase side chains, (meth)acrylate-based monomers selected from MEA, MEMA, PEGA, PEGMA and mixtures thereof, preferably selected from PEGA, PEGMA and mixtures thereof, and optionally one or more of the other non-nucleobase monomers as described in the above paragraph, preferably DEAEA, DEAEMA, or mixtures thereof.

Typically, the polymer comprises (meth)acrylate-based monomers with nucleobase side chains selected from thymine ethyl acrylate (ThEA), Thymine acetoxyethyl methacrylate (ThAcMA) and mixtures thereof, (meth)acrylate-based monomers with poly(ethylene glycol) side chains selected from PEGA, PEGMA and mixtures thereof, and optionally (meth)acrylate-based monomers selected from MEMA, MEA, DEAEA, DEAEMA, and mixtures thereof.

Typically, the polymer of the present invention comprises one or more of 1-90% MEA, 1-90% MEMA, 1-50% DEAEA, 1-50% PEGA, in combination with one or more of 1-50% ThEA and 1-50% ThAcMA. Typically, the polymer comprises 1-50% (preferably 20-50%) MEA, 1-50% (preferably 10-50%) MEMA, 1-50% (preferably 10-50%) DEAEA, DEAEMA or a mixture thereof, and 1-50% (preferably 1-20%) (meth)acrylate-based monomers with nucleobase side chains. The percentages of the polymer components described herein are all molar proportions with respect to the whole polymer.

Typically, the polymer comprises or consists of 1-50% MEA, 1-50% MEMA, 1-50% DEAEA, DEAEMA or a mixture thereof, 1-50% PEGA, PEGMA or a mixture thereof, and 1-50% (meth)acrylate-based monomers with nucleobase side chains, preferably selected from ThEA, ThAcMA, and mixtures thereof.

Typically, the polymer comprises or consist of 10-50% MEA, 10-50% MEMA, 10-50% DEAEA, DEAEMA or a mixture thereof, 1-25% PEGA, PEGMA or a mixture thereof, and 1-25% (meth)acrylate-based monomers with nucleobase side chains, preferably selected from ThEA, ThAcMA, and mixtures thereof.

Typically, the polymer comprises or consists of 20-50% MEA, 10-50% MEMA, 10-50% DEAEA, 1-15% PEGA, and 1-20% (meth)acrylate-based monomers with nucleobase side chains selected from ThEA, ThAcMA, and mixtures thereof.

Typically, the polymer corresponds to one of the following structures:

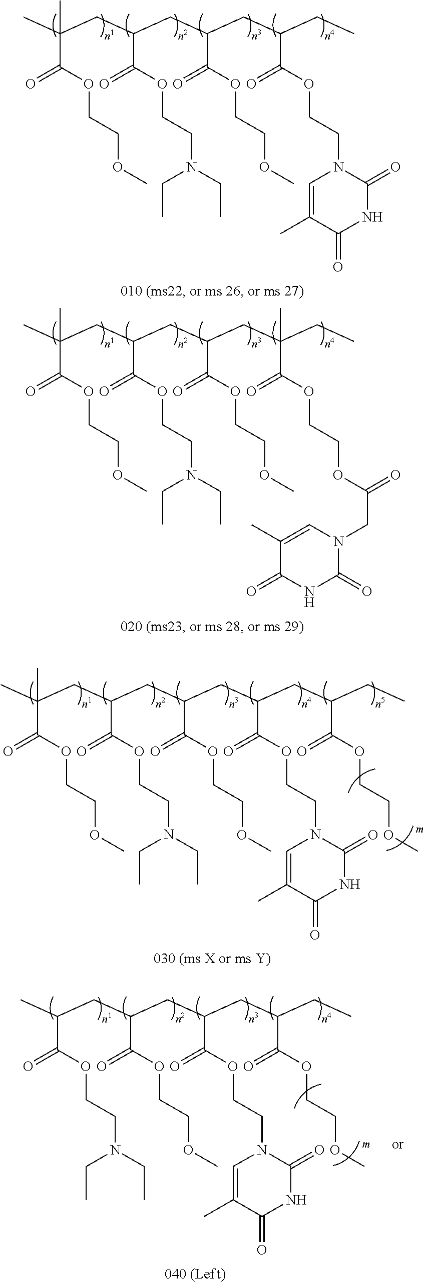

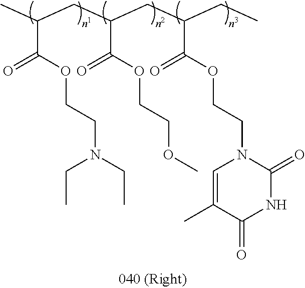

wherein the average value of m is from 1 to 50, preferably from 2 to 50, and values of n¹-n⁵ are selected to provide the polymer with the following compositions as indicated in Table 1 below:

TABLE 1

| | MEMA | DEAEA | MEA | ThEA | ThAcMA | PEGA |
|---|---|---|---|---|---|---|
| | (%) | (%) | (%) | (%) | (%) | (%) |
| Ms13 | 39 | 32 | 29 | — | — | — |
| Ms22 | 38 | 27 | 29 | 6 | — | — |
| Ms23 | 37 | 26 | 28 | — | 9 | — |
| Ms26 | 42 | 25 | 32 | 1 | — | — |
| Ms27 | 38 | 26 | 28 | 8 | — | — |
| Ms28 | 39 | 28 | 29 | — | 4 | — |
| Ms29 | 33 | 28 | 25 | — | 14 | — |
| MsY | 39 | 30 | 29 | 1 | — | 1 |
| MsX | 47 | 15 | 36 | 1 | — | 1 |
| 040(Left) | — | 10 | 88 | 1 | — | 1 |
| 040(Right) | — | 10 | 89 | 1 | — | — |

Ms13 polymer in Table 1 does not contain a nucleobase.

The composition of the polymer shown in Table 1 is determined by H-NMR (i.e. ¹H NMR). The percentages are molar proportions with respect to the whole polymer, calculated based on the following method. 5 mg (+0.5) polymer in 600 μL deuterated dimethyl sulfoxide was submitted for ¹H NMR analysis. The resulting spectrum was recorded using a Bruker AVA-500 at 500 MHz and 298K. The broad peak corresponding to 2H (3.80-4.30 ppm) on the carbon beta to the carboxylate on polymer side chains was set to represent 100% of the monomer components. This peak was shared by all monomers incorporated in the polymer. The spectrum was registered to its solvent peak and subsequently analysed for broad peaks at chemical shifts indicative of each type of polymerised monomer. Individual monomer components were identified in reference to pure monomer samples, integrated, and calculated as a proportion of all monomer components. For the polymers in Table 1, the identified peaks were grouped into the followings: all monomers, DEAEA, ThEA, and PEGA. Following integration of the DEAEA, ThEA, ThAcMA, and PEGA peaks and removing their percentage contribution from the ‘all monomers’ peak, the remaining contribution was split between MEMA and MEA in the proportion of the reaction feedstock. The percentage contribution for each of those monomers was then calculated based on integration of the proportion of signal from each monomer as part of the polymer signal as a whole. The method described herein can be referred to as ‘H-NMR Method’ (i.e. ‘¹H NMR Method’), and where applicable, it can be used to determine percentages of polymer components mentioned elsewhere in the specification.

Proportions of monomers used in the synthesis of the polymer are also provided (see Table 2). It will be understood that the proportions of the monomers used to prepare the polymer may be different to those derived from the resultant polymer as determined by H-NMR (i.e. ¹H NMR) in Table 1.

TABLE 2

| | MEMA | DEAEA | MEA | ThEA | ThAcMA | PEGA |
|---|---|---|---|---|---|---|
| | (%) | (%) | (%) | (%) | (%) | (%) |
| Ms13 | 40 | 30 | 30 | — | — | — |
| Ms22 | 38 | 28.5 | 28.5 | 5 | — | — |
| Ms23 | 38 | 28.5 | 28.5 | — | 5 | — |
| Ms26 | 39 | 30 | 30 | 1 | — | — |
| Ms27 | 36 | 27 | 27 | 10 | — | — |
| Ms28 | 39 | 30 | 30 | — | 1 | — |
| Ms29 | 36 | 27 | 27 | — | 10 | — |
| MsY | 38.5 | 30 | 29.5 | 1 | — | 1 |
| MsX | 51 | 10 | 37 | 1 | — | 1 |

Suitably, Mn value of PEGA used to prepare the polymer is about 480.

Any mixtures of the polymers described above can also be used in the present invention.

Typically, the polymer has a molecular weight from 1 kDa to 500 kDa. Typically, the molecular weight of the polymer is at least 10 kDa, more typically at least 20 kDa, still more typically at least 30 kDa, but not more than 100 kDa, typically not more than 60 kDa, more typically not more than 50 kDa.

The polymers may be prepared by known and effective methods to those skilled in the art. The polymer may be prepared by acrylate polymerisation. Suitably, the polymer is prepared by RAFT polymerisation. The method of preparing the apparatus May comprise an additional step of preparing the nucleobase-containing polymer, preferably by RAFT polymerisation.

In the context of the present invention, the substrate may be provided with the nucleobase-containing polymer. The polymer and the substrate may be as described above. Any mixtures of the polymers may also be used. The polymer may be firstly dissolved in a solvent. Thus, the method of preparing the apparatus may comprise steps of: (i) preparing a nucleobase-containing polymer preferably by RAFT polymerisation; (ii) optionally dissolving the polymer into a solvent; (iii) treating a substrate with the polymer, optionally the dissolved polymer; and (iv) treating the substrate provided with the polymer obtained in step (iii) with ethylene oxide. The solvent is preferably an organic solvent (e.g., tetrahydrofuran (THF)). Other examples of solvent include but are not limited to acetone, acetonitrile, butanol, cyclohexane, diethylene glycol, diethyl ether, diglyme, 1,2-dimethoxy-ethane, ethylene glycol, glycerin, heptane, hexane, chloroform, dichloromethane, dimethyl sulfoxide, ethyl acetate, methylene chloride, petroleum ether, propanol and triethyl amine. Suitably, the substrate is formed of a different material (i.e a material that is not the nucleobase-containing polymer). It is understood that said substrate does not dissolve in the solvent and the nucleobase-containing polymer suitably dissolves in the solvent. The polymer concentration is suitably at least 0.01 w/v % (weight per volume percentage concentration, 0.01 g/100 mL), or at least 0.1 w/v %, or at least 0.5 w/v %, or at least 1 w/v %, optionally not more than 10 w/v %, or not more than 5 w/v %, or not more than 1 w/v %, or not more than 0.5 w/v %. Typically, a dissolved polymer is used to treat the substrate.

Treatment with Ethylene Oxide (EtO)

It will be understood that treating the substrate with ethylene oxide is beneficial. Normally, the treatment of the present invention is adapted to be compliant with ISO 11135-1:2007 on the standardization of ethylene oxide sterilization of healthcare products. Herein, by ‘ethylene oxide’ is meant a cyclic ether which is normally a colourless gas (e.g. at 1 atm and at ambient temperature which is 20-25° C.).

When ethylene oxide is used to treat the substrate and/or the polymer, it can effectively kill the microorganisms that would otherwise pose a health risk to the users and subjects (e.g. patients). In other words, ethylene oxide treatment can provide effective sterilisation (possibly due to its excellent microbicidal activity). Moreover, it is surprisingly found that ethylene oxide can preserve or boost the capture as well as recovery of nucleic acid. This is in stark contrast to other sterilization methods (e.g. gamma radiation) which negatively affect the efficacy of the apparatus.

Without wishing to be bound by any theory, it is believed that ethylene oxide preserves and promotes interaction between the nucleic acid and the apparatus. It is possible that ethylene oxide creates a beneficial change in the polymer and/or the substrate (e.g. the substrate provided with the polymer) to improve interaction with the nucleic acid. It is possible that ethylene oxide promotes compositional change of the substrate and/or the polymer (e.g., the substrate provided with the polymer). Alternatively or additionally, one or more surface properties of the substrate and/or polymer may be modified to preserve or promote said interaction. It is possible that a plurality of ethylene oxide units graft to the polymer and/or the substrate (e.g. the substrate provided with the polymer) to increase their hydrophilicity. This increased hydrophilicity may occlude other components from the sample from interacting with the polymer and/or substrate, and allow hydrophilic nucleic acids (e.g., cfDNA) to reach the apparatus more quickly and interact with the polymer and/or the substrate more successfully. For example, it is possible that ethylene oxide creates a compositional change in the polymer to make it more hydrophilic, thus pushing away the plasma proteins but draws in hydrophilic DNAs (e.g. cfDNAs); and/or the ethylene oxide introduces a molecular crowding that promotes DNA (e.g. cfDNA) adsorption onto surfaces of the polymer and/or the substrate. Thus, it is possible that ethylene oxide units take the form of polyethylene oxides (PEOs) which exhibit a ‘molecular crowding’ effect that brings nucleic acids into close contact with the polymer and/or substrate to increase efficacy of the apparatus. The conditions of ethylene oxide can be further tuned (e.g. enhanced) to further improve the nucleic acid recovery. It is believed that under enhanced conditions, ethylene oxide can modify the substrate and/or the polymer (e.g. the substrate provided with the polymer) to a greater extent in favour of interaction with nucleic acids. The resultant apparatus is not only sterilised, but also effective in capturing as well as recovering nucleic acid (e.g. cf DNA).

In the context of the present invention, the substrate and/or the polymer (e.g. the substrate provided with the polymer as a whole) may be modified by ethylene oxide. It is possible that at least one aspect of the substrate and/or the polymer (e.g. the substrate provided with the polymer) is modified by ethylene oxide. Possibly, both the substrate and the polymer (e.g. the substrate provided with the polymer) are modified, or at least the polymer is modified. Possibly, the substrate provided with the polymer is modified.

Typically, the substrate and/or the polymer (e.g. the substrate provided with the polymer) is treated (e.g. modified) by ethylene oxide applied from an ethylene oxide/diluent mixture, wherein optionally the ethylene oxide concentration is at least 100 mg/L, or at least 200 mg/L, or at least 300 mg/L, or at least 400 mg/L, or at least 800 mg/L, or at least 1000 mg/L. But the ethylene oxide concentration may be optionally not more than 2000 mg/L, or not more than 1600 mg/L, or not more than 1500 mg/L, or not more than 1200 mg/L, or not more than 1000 mg/L. For example, the ethylene oxide concentration may be from 300 mg/L to 2000 mg/L, or from 400 mg/L to 1200 mg/L, or from 800 mg/L to 1000 mg/L. Herein, diluent refers to liquid or gas, preferably gas. Suitable examples of diluents include but are not limited to carbon dioxide (CO₂) and nitrogen (N₂) gas.

Additionally and optionally, ethylene oxide or ethylene oxide/diluent mixture may be applied at a temperature of at least 15° C., or at least 20° C., or at least 30° C., or at least 40° C., or at least 45° C., or at least 50° C. Ethylene oxide or ethylene oxide/diluent mixture may optionally be applied at a temperature of no more than 90° C., or no more than 80° C., or no more than 70° C., or no more than 60° C., or no more than 55° C. For example, ethylene oxide or ethylene oxide/diluent mixture may be applied at a temperature of from 30° C. to 70° C., or from 40° C. to 60° C., or from 40° C. to 55° C.

Additionally and optionally, ethylene oxide or the mixture may be applied at a humidity of at least 30%, or at least 40%, or at least 45%, or at least 50%, or at least 60% or at least 65%, or at least 70%, or at least 75%. It may be that the humidity is not more than 90%, or not more than 85%, or not more than 80%. For example, the ethylene oxide or the mixture may be applied at a humidity of at least 30% or at least 50%.

Additionally and optionally, ethylene oxide or ethylene oxide/diluent mixture may be applied for a period of at least 1 hour, or at least 2 hours, or at least 2.5 hours, or at least 3 hours, or at least 4 hours, or at least 6 hours, or at least 7 hours, or at least 10 hours, or at least 12 hours.

Optionally, the substrate and/or the polymer (e.g. the substrate provided with the polymer) is treated (e.g. modified) with ethylene oxide applied from an ethylene oxide/diluent mixture in which the ethylene oxide concentration is at least 300 mg/L, and wherein the treatment is for a period of at least 2 hours, at a temperature of at least 40° C., and at a humidity of at least 30%.

Optionally, the substrate and/or the polymer (e.g. the substrate provided with the polymer) is treated (e.g. modified) with ethylene oxide applied from an ethylene oxide/diluent mixture in which the ethylene oxide concentration is at least 400 mg/L, and wherein the treatment is for a period of at least 2 hours, at a temperature of at least 45° C., and at a humidity of at least 30%.

Optionally, the substrate and/or the polymer (e.g. the substrate provided with the polymer) is treated (e.g. modified) with ethylene oxide applied from an ethylene oxide/diluent mixture in which the ethylene oxide concentration is at least 800 mg/L, and wherein the treatment is for a period of at least 7 hours, at a temperature of at least 45° C., and at a humidity of at least 50%.

In most preferred embodiments, the substrate and/or the polymer (e.g. the substrate provided with the polymer) is treated (e.g. modified) by ethylene oxide applied from an ethylene oxide/diluent mixture under enhanced conditions, wherein the ethylene oxide concentration ranges from 800 mg/L to 1200 mg/L (e.g. 800 mg/L, 1000 mg/L), the temperature ranges from 40° C. to 60° C. (e.g., 45° C., 50° C.), the humidity ranges from 50% to 90% (e.g., 50%, 75%), and the time period of modification ranges from 5 hours to 9 hours (e.g. 7 hours). In the same or other embodiments, the time period can be prolonged, for example to at least 12 hours.

Any of the above-described treatments with ethylene oxide can be optionally repeated, once, or at least 2 times.

Preferably, the polymer and/or the substrate (e.g. the substrate provided with the polymer) of the present invention is treated by ethylene oxide, wherein the ethylene oxide is applied from an ethylene oxide/diluent mixture in which the ethylene oxide concentration is at least 1000 mg/L, optionally the temperature of treatment is at least 15° C. (e.g. at least 50° C.), further optionally the humidity of the treatment is at least 75%, and still further optionally the duration of the treatment is at least 7 hours. It may be that the ethylene oxide concentration is at least 100 mg/L, optionally the temperature is at least 15° C. or at least 40° C., further optionally the humidity is at least 40% and still further optionally the duration is at least 3 hours. It may be that the ethylene oxide concentration is at least 100 mg/L, optionally the temperature is at least 15° C., further optionally the humidity is at least 30%, and still further optionally the duration is at least hour. It may be that the upper limit for any of the described conditions in this paragraph is not more than 2000 mg/L (for an ethylene oxide concentration), and/or not more than 90% (for humidity).

In the context of the present invention, it may also be that the substrate and/or the polymer (e.g. the substrate provided with the polymer) is treated (e.g. modified) by ethylene oxide, resulting in the substrate and/or the polymer (e.g. the substrate provided with the polymer) comprising one or more ethylene oxide (i.e. ethylene glycol) units. At least some of said ethylene oxide units may be in the form of polyethylene oxides (PEOs). It may be that the substrate comprises one or more PEOs, and optionally a plurality of single ethylene oxide units. Alternatively and additionally, it May be that the nucleobase-containing polymer comprises one or more PEOs, and optionally a plurality of single ethylene oxide units. The ethylene oxide units (e.g. the PEOs and/or the single ethylene oxide units) may be grafted onto (e.g., anchored to) the substrate and/or polymer. As previously discussed, it may be that the ethylene oxide treatment creates a compositional change in the nucleobase-containing polymer, thereby improving the efficiency of DNA recovery. It may be that the substrate and/or the polymer is modified with one or more ethylene oxide units.

It may be that the nucleobase-containing polymer per seis treated with ethylene oxide. It may be that the nucleobase-containing polymer per se is treated with ethylene oxide such that one or more changes happen to the polymer. For example, one or more ethylene glycol (i.e. ethylene oxide) units may be provided to the nucleobase-containing polymer. It may be that the resultant nucleobase-containing polymer comprises one or more ethylene glycol units. The nucleobase-containing polymer May be treated with ethylene oxide according to the treatment method described herein. The nucleobase-containing polymer per se (i.e. the nucleobase-containing polymer prior to ethylene oxide treatment) may be as described herein (e.g. as described hereinbefore). The resultant nucleobase-containing polymer in itself is believed to be novel. Without wishing to be bound by any theory, it is believed that this novel polymer improves DNA recovery in comparison to the nucleobase-containing polymer without ethylene oxide treatment.

It may be that the nucleobase-containing polymer is treated by ethylene oxide, and then is provided to the substrate described hereinbefore (e.g. is coated onto the substrate described hereinbefore). For example, the substrate described hereinbefore may have a specific surface area of at least 10 cm²/cm³, and/or is formed from polyurethane, optionally a polyurethane sponge. The substrate described hereinbefore may be sterilised (e.g. sterilised by ethylene oxide); and the sterilised substrate is provided (e.g. coated) with the ethylene oxide treated polymer. Alternatively or additionally, the substrate provided (e.g. coated) with the ethylene oxide treated polymer may be treated with ethylene oxide, thereby sterilising said substrate provided with said polymer.

As above, the method of preparing the apparatus may comprise: providing a substrate, wherein the substrate is the nucleobase-containing polymer; treating said polymer with ethylene oxide; and providing the treated polymer to a further substrate, wherein optionally the further substrate is sterilised, further optionally sterilised by ethylene oxide. Additionally and optionally, after providing the treated polymer to the further substrate, this method comprises treating the further substrate provided with the polymer with ethylene oxide, thereby sterilising said substrate provided with said polymer.

Typically, the substrate and/or the polymer is treated (e.g. modified) with ethylene oxide in an ethylene oxide sterilizer. Typically, the substrate and/or the polymer is cleaned of contaminants prior to ethylene oxide treatment. Herein, contaminants refer to stain, soil, grease, dirt, dust, smut, soot, and the like. Typically, the substrate and/or polymer is cleaned by water.

Optionally, the treatment with ethylene oxide can be repeated, preferably repeated once or twice. Suitably, the treatment is repeated once.

### Apparatus

In the context of the present invention, the apparatus so prepared can be a sample (e.g. fluid) collecting apparatus, for example, a fluid collector. The apparatus can be a fluidic apparatus, for example, a flow cell. The apparatus can be an apheresis apparatus (i.e. an apparatus for use in an apheresis procedure). Suitably, the apparatus is a fluidic as well as apheresis apparatus. Suitably, the apparatus is an extracorporeal apparatus (e.g. an apheresis and extracorporeal apparatus). Herein, ‘apheresis’ is used to mean a technology in which a fluid (e.g. blood) obtained from a subject (e.g. a patient) is passed through an apparatus that captures at least one component of the fluid, and one or more of the other components of the fluid are returned to circulation. The returned fluid may contain reduced level of the captured component or not contain said component (e.g. the component is completely removed from the fluid). The captured component may be nucleic acid, preferably cfDNA, more preferably ctDNA. The apheresis can be partly or completely extracorporeal. The apheresis can be at least partly intracorporeal. It is also possible for the apheresis to be partly extracorporeal and partly intracorporeal. It will be understood that the apheresis may contain a step of returning one or more of the other components of the fluid to the subject (e.g. returning the other components of the blood to a patient). Additionally or alternatively, the apheresis may contain a step of obtaining the fluid from the subject (e.g. obtaining blood from a patient). The apparatus of the present invention may be configured to conduct the apheresis procedure as described herein. The apparatus may comprise a means (e.g. an injector) for returning one or more of the other components to the circulation and/or to the subject (e.g. patient). Additionally or alternatively, the apparatus may comprise a means (e.g. an extractor) for withdrawing a sample (e.g. blood) from the subject (e.g. patient). The apparatus may be configured to conduct the apheresis procedure (e.g. a part of the apheresis procedure) that happens outside a human or animal body.

It will be understood that the apparatus of the invention may not be limited to an apheresis apparatus. For example, the apparatus may be configured to capture nucleic acid from a urine sample. The other components of the urine may be suitably discarded instead of being returned to the patient. The apparatus may comprise a means for retaining (e.g. an enclosure) the other components prior to their disposal. The apparatus may comprise a means for releasing the other components (e.g. an outlet). The means for releasing the other components may be connected or connectable to a disposal device.

It will be further understood that the apparatus may be configured to capture nucleic acids from a bodily fluid extracorporeally. For example, the apparatus may comprise (e.g be) a fluid collector and the substrate provided with the nucleobase-containing polymer may be received or receivable in said collector. The collector may optionally comprise (e.g. be) a collection tube (e.g. blood collection tube), or a collection pot (e.g., a urine collection pot), or any container suitable for collecting fluid as well as receiving the substrate provided with the nucleobase-containing polymer. Said substrate may be suitably received or receivable in said tube, said pot, or said container. The collector may optionally have a lid (to prevent the spillage of the fluid). The collector may be connected or connectable to one or more catheters. The catheters may be configured to put fluid into the subject (e.g. the body of the patient) or take it out. The apparatus may further comprise a receptacle (e.g. a sealable plastic bag), wherein the fluid collector is contained within said receptacle. The apparatus may further comprise one or more printed or written labels. The labels may contain the sample (e.g. blood sample or urine sample) and/or the subject (e.g. the patient) information. The labels may also be in any medium capable of storing the information and communicating it to the user of the apparatus.

The apparatus may be configured to adjust one or more parameters such as temperature, volume, pressure, and fluidic speed of the sample. The apparatus May comprise one or more controllers to adjust those parameters (e.g. a temperature controller). The apparatus may comprise a temperature sensor, or a pressure sensor, or a speed sensor, or a volume sensor, or combinations thereof. Said sensors may be optionally incorporated into said controllers. For example, the temperature sensor may be incorporated into the temperature controller. The apparatus may comprise a container, wherein the substrate provided with the nucleobase-containing polymer is received or receivable within said container. The container may be made of suitable materials such as polycarbonate, polytetrafluoroethylene, polypropylene, glass, plastic (e.g., high/low density polyethylene (H/LDPE), polystyrene (PS)), and recycled plastic. Preferably the container is made of recycled plastic. The container may comprise a lid to retain the substrate. Optionally, the lid is made of the same material as the container. In the context of the present invention, the container may optionally be the fluid collector as described herein.

The apparatus may be configured to separate one or more of the other components from nucleic acids of the sample. The apparatus may comprise means for separating (e.g. a separator) other components from nucleic acids of the sample. The apparatus may comprise a sample inlet and a sample outlet. The apparatus may comprise a sample input path (connected or connectable to the sample inlet), and a sample output path (connected or connectable to the sample outlet), and the input path to the output path is extendable or extends over the nucleobase-containing polymer. The input path to the output path may extend over or be extendable over the container described herein, wherein the substrate provided with the nucleobase-containing polymer May be received or receivable in said container. The apparatus may comprise an input path comprising an inlet connected or connectable to an inlet tube and an output path comprising an outlet connected or connectable to an outlet tube. The inlet tube may be connected or connectable to a first device configured to withdraw the sample (e.g. blood) from a subject (e.g. a patient). The outlet tube may be connected or connectable to a second device configured to return the sample with reduced level of nucleic acids (e.g. cfDNA) to the subject. The first device and the second device may be the same (e.g. a device comprising a withdrawal path and a return path) or different devices. Said devices may be part of the apparatus. Typically, the apparatus described above (e.g. an apheresis apparatus) is configured to form at least part of a loop to withdraw the sample from the subject and to return the sample with reduced amount of nucleic acid (e.g. cfDNA) to the same subject.

In a second aspect of the invention, there is provided an apparatus prepared according to the method of the first aspect. The characteristics of the apparatus (e.g. the substrate, the polymer, the ethylene oxide treatment) may be as described herein. Further, the apparatus may be provided as a product wherein said apparatus is contained within a packaging, and the product preferably comprises instructions for use. Typically, the packaging comprises a primary packaging within which the apparatus is contained. Suitable examples for a primary packaging are pouches (e.g. Tyvek® pouches). Optionally, the packaging may comprise a secondary packaging, within which the primary packaging is contained. Suitable examples for a secondary packaging are cartons or plastic boxes. Suitably, at least one of the packaging described above can protect the apparatus from light (e.g., direct sunlight), moisture, physical and/or chemical damages and contaminants. The instructions may give detailed information for the steps of using the apparatus in accordance with the second aspect of the invention. The instructions may be supplied in the printed or written form of a label, a booklet, a brochure, a scannable code (e.g. a barcode), or a leaflet. The instructions may also be printed or written on at least one packaging of the product.

### Methods

In the third aspect of the application, there is provided a method for preserving or improving capture of a nucleic acid from a sample, comprising the steps of: (i) providing a sample comprising a nucleic acid; (ii) providing an apparatus according to the second aspect of the invention, (iii) providing the sample of step (i) to the polymer comprised in the apparatus of step (ii), and (iv) capturing the nucleic acid by the polymer. The EtO treatment sterilises the apparatus and leads to preservation or improvement of its efficacy.

The method may be optionally repeated (e.g. once or twice) to further increase the nucleic acid supply for further analysis. Typically for one single operation, the method 32 can capture from 10% to 90% nucleic acid contained in the sample, more typically from 30% to 85%, still more typically from 50% to 80%, and optionally the recovery rate of the nucleic acid is from 5% to 90%, typically from 10% to 80%, more typically from 15% to 70%, most typically from 50% to 70%.

In the present invention, the sample containing the nucleic acid may be provided to the polymer under the condition that allows the polymer to capture the acid. Typically, the sample and the polymer (e.g., the substrate provided with the polymer) are incubated, optionally at a temperature of at least 4° C., more typically at least 20° C., still more typically at least 30° C., but not more than 40° C. A suitable example of the temperature is about 37° C. (i.e., equivalent to human body temperature). Typically, the incubation period is at least 10 seconds, more typically at least 20 seconds, still more typically at least 1 minute, but not more than 2 hours, typically not more than 1 hour, more typically not more than 50 minutes, most typically not more than 40 minutes. These incubation periods may be suitable for an apheresis procedure (e.g. an apheresis apparatus). It is also possible that the incubation period is at least 1 hour or at least 3 hours, or at least 6 hours, or at least 12 hours, but not more than 7 days, or not more than 2 days, or not more than 24 hours. The longer incubation period is typical for an apparatus that comprises (e.g. is) a fluid collector. Typically, the sample is flowed over the polymer, optionally at a flow rate of at least 20 mL/minute, more typically at least 30 mL/minute, but not more than 100 mL/minute, or not more than 80 mL/minute, or not more than 70 mL/minute. Typically, the sample and the polymer (e.g., the substrate provided with the polymer) are incubated in an incubation composition. The composition may be a solution. The composition may be a blood (e.g. whole blood). The composition (e.g. blood) may or may not be diluted. For instance, in the apheresis procedure, it is possible to introduce an anticoagulant solution (e.g. continuously or intermittently or at least once) to the blood. This can reduce or prevent blood clotting.

The method may further comprise one or more of the following steps of: a. separating the nucleic acid from the sample optionally without the use of centrifugation or pre-filtration; b. washing a polymer-nucleic acid complex to remove one or more other components from the sample; c. releasing some of or all of the nucleic acid from the polymer; d. characterising the nucleic acid; e. sequencing the nucleic acid; and f. identifying a disease state based on the characterisation of the nucleic acid.

Typically, the captured nucleic acid stays stabilized. Herein, by ‘stabilized’ is meant that the nucleic acid is not degraded or undergoes any changes that can affect the chemical nature of the acid. The nucleic acid may be stabilized for at least 2 days, typically at least 4 days, more typically at least 5 days and most typically at least 7 days. The nucleic acid may stay stabilized (e.g. preferably on the substrate, or by the substrate) during and/or after washing to remove other components from the sample (i.e. step b). The stabilized nucleic acid may also be immobile on the substrate. The captured nucleic acid may be transported or stored for subsequent analysis, with or without the substrate. The nucleic acid (without the substrate) or the nucleic acid bound to the substrate may be used for the subsequent analysis.

Typically, the polymer-nucleic acid complex is washed by a saline. Typically, the saline has a neutral pH (i.e. pH from 6 to 8). A suitable example of the saline is phosphate-buffered saline (PBS).

Typically, at least some of if not all of the nucleic acid is released from the polymer with an elution buffer. Typically, the elution buffer has an ionic strength equivalent to or higher than that of 600 mM sodium chloride solution. The elution buffer may have a pH from 4 to 10, typically from 5 to 9, more typically from 6 to 9, most typically from 8 to 9. It is found that the elution buffer with high ionic strength and/or alkaline pH leads to high efficiency of nucleic acid recovery. The recovered nucleic acid can then be used for subsequent analysis, with or without purification. The above-described eluting procedure can be optionally repeated to afford more nucleic acid, preferably repeated once or twice.

Typically, characterisation of the nucleic acid may include one or more activities of: determining a concentration, a quality metric, a physical mapping, a sequence content, an epigenetic information, SNP, a haplotype, an RFLP, sizing, and a copy number variant.

Typically, sequencing the nucleic acid may be via Next Generation Sequencing. The captured nucleic acid may be used in one or more PCR methods. It may be that the captured nucleic acid is used for sequencing and/or one or more PCR methods.

Identification of disease states may involve purification and optionally identification or quantification of nucleic acid, preferably cfDNA. Suitably the identification may comprise labelling polynucleotides of cfDNA to identify or quantify the cfDNA. A fluorophore, a quantum dot, a dendrimer, a nanowire, a bead, a hapten, a streptavidin, an avidin, a neutravidin, a biotin, and a reactive group a peptide, a protein, a magnetic bead, a radiolabel, or a non-optical label may be applied to the nucleic acid. In some embodiments, a label can be a fluorophore or a quantum dot.

Typically, the method of the third aspect is part of an apheresis method optionally with the nucleic acid comprised in a blood sample, or the method is an extracorporeal method optionally with the nucleic acid comprised in a urine or a blood sample. The method may also be an extracorporeal method optionally with the nucleic acid comprised in a bodily content (e.g. bodily fluid). Typically the bodily content is outside a human or animal body. The bodily content (e.g. 10 mL) can be, for example, blood or plasma, preferably plasma. Preferably, the method is part of an apheresis method, and preferably said part happens outside human and animal bodies. Since the volume of blood drawn from a subject (e.g. a patient) is limited, it is highly beneficial to capture the nucleic acid (e.g. cfDNA) from the blood, and then return the other blood components back to the subject. In this way, the apheresis method can harvest large quantity of nucleic acid (e.g. cfDNA) without adversely affecting the blood level of the subject. Typically, the polymer and the substrate provided with the polymer are haemocompatible. In other words, they may cause minimal coagulation of the blood 12 during the apheresis procedure.

The apheresis method typically comprises steps of (i) providing a nucleic acid-containing sample (e.g. blood) to the polymer comprised in the apparatus of the invention, (ii) capturing the nucleic acid by the polymer and (iii) returning the other components of the sample to a subject (e.g. a patient). Preferably, the nucleic acid is a cfDNA. It will be understood that the returned sample contains reduced a level of nucleic acid. The reduction may be at least 10% by weight, typically at least 20%, more typically at least 40%, still more typically at least 60%, most typically at least 70%. Typically, at least 60% by weight of the other components are returned to the subject, more typically at least 70%, still more typically at least 90%, most typically 100%. The method may be an apheresis method. The method may be part of an apheresis method. The method may be part of an apheresis method wherein said part happens outside a human or animal body. For example, the method may comprise (e.g. consist of) the steps (i) and (ii).

Optionally, the apheresis method may comprise one or more of the steps (a) to (f) as described above. Optionally, the apheresis method may be repeated (e.g. once or twice).

### Use of Ethylene Oxide

In a fourth aspect of the invention, there is provided use of ethylene oxide to preserve or improve capture of a nucleic acid from a sample, wherein ethylene oxide is used to treat a substrate and/or a polymer. Said substrate and polymer are comprised in an apparatus. The ethylene oxide treated apparatus is subsequently used to capture a nucleic acid comprised in a sample. Said substrate is a nucleobase-containing polymer or is provided with said polymer. The use may possess characteristics as described in the method of the third aspect of the invention.

In a fifth aspect of the invention, there is provided use of ethylene oxide to manufacture an apparatus comprising a substrate, wherein the substrate is a nucleobase-containing polymer or is provided with said polymer, and said substrate and/or polymer is treated (e.g. modified) with ethylene oxide.

In the fourth and the fifth aspects, the characteristics of the substrate, the polymer and the treatment with ethylene oxide may be as described in the first aspect or described herein. Optionally, the treatment by ethylene oxide may be repeated, preferably repeated once.

Further, the present invention may provide use of ethylene oxide to sterilise the apparatus. The apparatus comprises a substrate which is a nucleobase-containing polymer or is provided with said polymer. The substrate and/or polymer is sterilised by ethylene oxide. The apparatus, the substrate, the polymer and the ethylene oxide treatment may be as described herein.

Any feature of one aspect of the present invention may be utilised in any other aspect of the invention where appropriate. Any feature of a particular embodiment may be utilized in any other embodiment of the invention where appropriate. Features, integers, characteristics, or groups described in conjunction with a particular aspect, embodiment, or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The word ‘comprising’ is intended to mean ‘including’ but not necessarily ‘consisting of’ or ‘composed of’. In other words, the listed steps or options need not be exhaustive. Throughout the description and claims of this specification, the singular encompasses the plural unless the context otherwise requires, and vice versa. In particular, where the indefinite article is used, the specification is to be understood as contemplating plurality as well as singularity, unless the context requires otherwise. The examples and embodiments given in the description above are intended to clarify the invention but not to limit the invention. All percentages are weight percentages based upon the total weight of relevant materials unless otherwise indicated. Except in the operating and comparative examples, or where otherwise explicitly indicated, all numbers in this description indicating amounts of material or conditions of reaction, physical properties or materials and/or use are to be understood as modified by the word ‘about’. Numerical ranges expressed in the format ‘from x to y’ are understood to include x and y, unless specified otherwise. When for a specific feature multiple optional ranges are described, it is understood that all ranges combining the different endpoints are also contemplated.

### EXAMPLES

The present invention will now be illustrated with reference to the following non-limiting Examples.

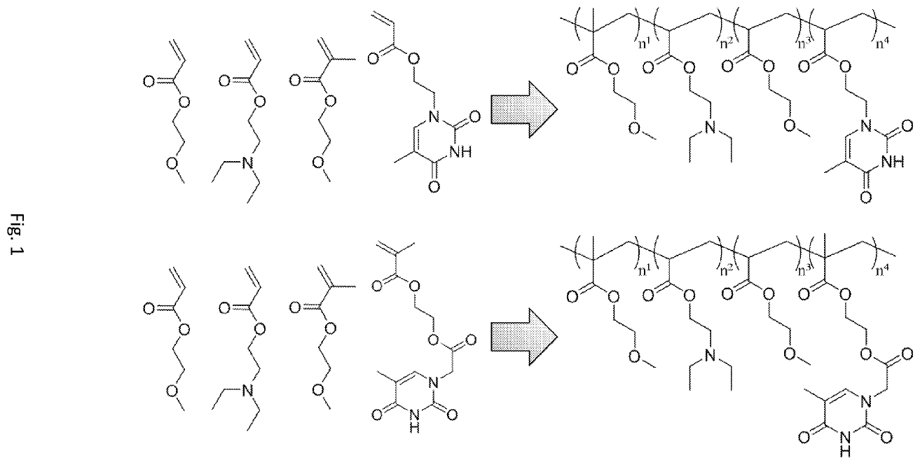

FIG. 1: illustrates the synthesis of nucleobase-containing polymers comprising thymine derivatives.

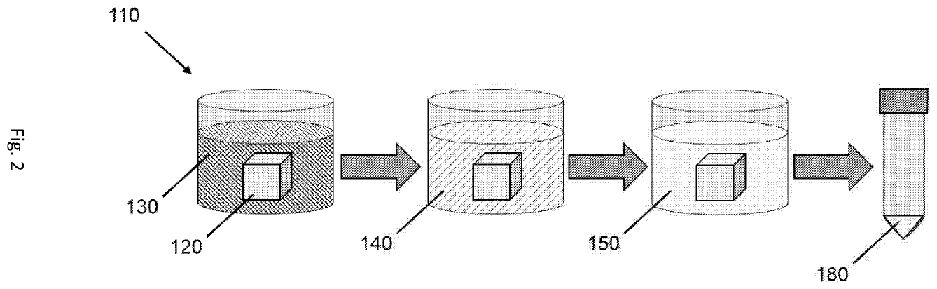

FIG. 2: illustrates an overview of cfDNA capture using a substrate coated with nucleobase-containing polymer and treated by ethylene oxide.

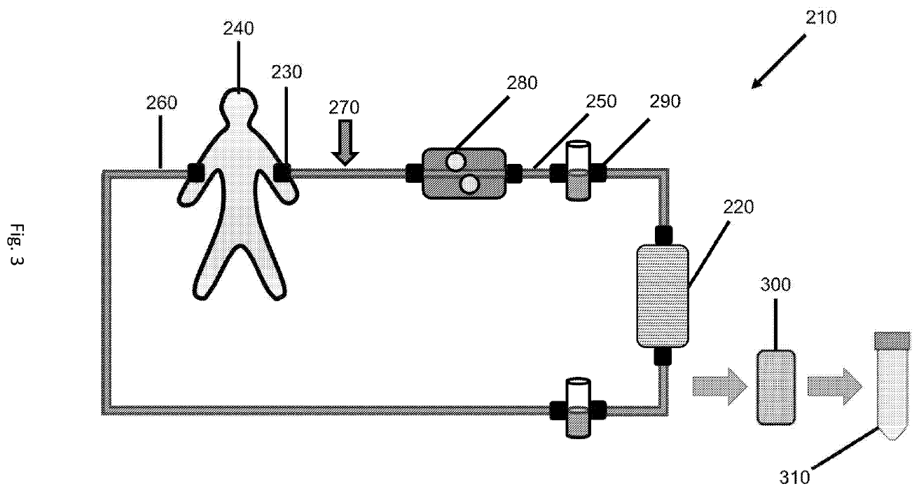

FIG. 3: illustrates an overview of cfDNA capture using an apheresis method.

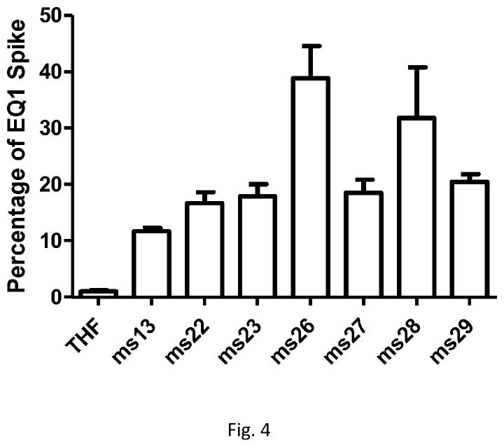

FIG. 4: illustrates the sequestering of radiolabelled cfDNA from whole blood using a range of polymers and THF.

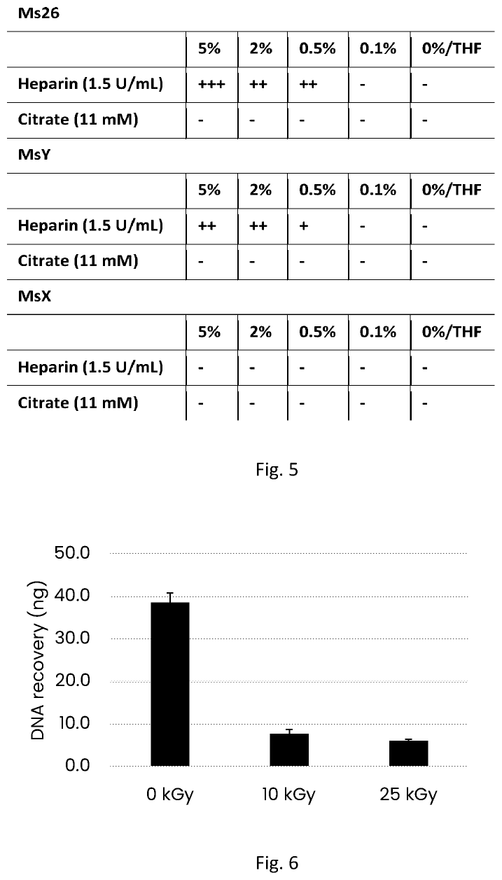

FIG. 5: illustrates the haemocompatibility of a range of polymer-coated sponges.

FIG. 6: illustrates the DNA recovery after gamma radiation.

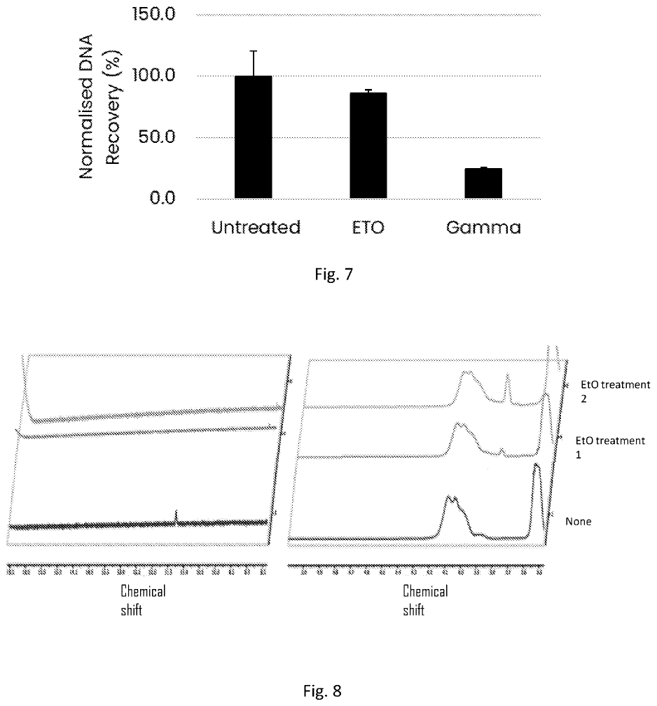

FIG. 7: illustrates the DNA recovery after gamma radiation and EtO treatment

FIG. 8: illustrates the NMR analysis of Ms26 after various EtO treatments

### EXAMPLE 1—POLYMER SYNTHESIS

FIG. 1 illustrates the synthesis of nucleobase-containing polymers, wherein polymerisation reactions of acrylate/methacrylate monomers result in those polymers.

The polymers used were typically synthesised on a 1 g scale. Monomers were passed through basic alumina columns to remove inhibitors. These purified monomers were combined in the presence of a chain transfer agent (2-Cyano-2-propyl dodecyl trithiocarbonate) and initiator (2,2′-Azobis(2-methylpropionitrile)) at a typical ratio of 1000:5:1, or 1500:5:1 in a microwave vial and dissolved in DMSO to a final reaction concentration of 5 molar. The reactions were degassed with N₂ for 30 minutes. Polymerisations were stirred at 60° C. under N₂ for about 48 hours, or up to 7 days. The reactions were stopped by cooling with dry ice and acetone, and exposed to air. The polymers were purified by precipitation first into water, then three times by dissolving in THF and precipitated in hexane. The polymers were then dried of solvent in a vacuum oven (40° C.).

Proportions of monomers that were used in the synthesis of the polymers are provided in Table 2 hereinbefore. The polymers are Ms13, Ms22, Ms23, Ms26, Ms27, Ms28, Ms29, MsY and MsX.

¹H-NMR was used to measure the composition of those polymers. The results are as provided in Table 1 hereinbefore. In detail, ‘H-NMR Method’ can be used. The characterisation was undertaken using a process wherein 5 mg of dried polymer was dissolved in 600 μL DMSO-d6 and submitted for compositional analysis using ¹H-NMR, recorded using a Bruker AVA-500 at 500 MHz and 298K. The broad peak corresponding to 2H (3.80-4.30 ppm) on the carbon beta to the carboxylate on polymer side chains was set to represent 100% of the monomer components. This peak is shared by all monomers incorporated in the polymer. Individual monomer components were identified in reference to pure monomer samples, integrated, and calculated as a proportion of all monomer components.

### EXAMPLE 2—SUBSTRATES AND ETHYLENE OXIDE TREATMENT

The polymers obtained in Example 1 could be directly used as substrates. Alternatively, the substrates used were polyurethane sponges. The polymers of Example 1 were dissolved in THF, and then coated onto those sponges. Scanning electron microscopy (SEM) verified that the polymers generally coated on the surfaces of the sponges by forming one or more layers on said surfaces.

Subsequently, some of the substrates (i.e. polymers or substrates provided with polymers) were subjected to ethylene oxide treatment in an ethylene oxide steriliser, in accordance with the present invention.

### EXAMPLE 3—USE OF THE SUBSTRATE TO CAPTURE CFDNA

FIG. 2 illustrates a process of capturing cfDNA from a solution 110 by using a substrate (120) provided with a nucleobase-containing polymer. The solution is substantially undiluted whole blood 130, which comprises cfDNA. The cfDNA in the blood would then bind to the polymer on the substrate. A wash buffer 140 would then be used to remove residual material from the whole blood while retaining the cfDNA on the substrate. The wash buffer in this example would comprise phosphate buffered saline (PBS). An elution buffer 150 would then be provided to elute the cfDNA from the substrate. The elution buffer was 100 mM Tris-HCl pH 8.5, 1250 mM NaCl (high salt buffer with a NaCl concentration >600 mM). The elution, as stored in a tube 180, may then be purified or concentrated using conventional means to produce a cfDNA sample which may then be characterised.

### EXAMPLE 4—USE OF AN APHERESIS PROCEDURE TO CAPTURE CFDNA

FIG. 3 describes an apheresis procedure (210), wherein whole blood (230) is removed from a subject (240) and provided to a system comprising a sampling loop (250) before being returned (260) to the subject. To the whole blood may be added an anticoagulant (270) to prevent blood clotting while it is extracorporeal. The sampling loop comprises a nucleobase-containing polymer bound to a substrate (220). The sampling loop further comprises a peristaltic pump (280), and bubble trap or traps (290). When whole blood passes through the substrate, cfDNA may bind to the polymer and other whole blood components may not. When sufficient cfDNA has been bound to the polymer, the substrate is removed from the sampling loop (300). The substrate is suitably washed such that the cfDNA is retained while other blood components (if bound) are removed from the substrate. The cfDNA is then eluted under suitable conditions to provide a concentrated sample of cfDNA for characterisation (310).

### EXAMPLE 5—SEQUESTERING OF RADIOLABELLED CFDNA

FIG. 4 shows that the polymers of Example 1 and THF were screened for DNA recovery from whole blood (pig) spiked with DNA (³H-labelled chromatin: mouse mononucleosomal, 160 bp). Polyurethane sponges were used as substrates. Some substrates were coated with the polymers of Example 1. A control substrate was coated with THF. Each coated substrate was incubated in the spiked blood for 30 minutes at 37° C. The sponges were then removed from the spiked blood and washed with PBS before elution of the bound DNA from the polymers using an elution buffer consisting of 0.1M sodium acetate, pH 5, 0.6M NaCl, 0.15% Triton X-100 (EQ1 Buffer, Invitrogen™). The concentration of labelled DNA was quantified using liquid scintillation counting. Because the measured 3H signal can be influenced by the composition of the measurement solution, the results are represented as proportions of the signal measured from the quantity of DNA spiked into blood, in EQ1.

The result shows the polyurethane sponges coated with the polymers of Example 1 recovered more cfDNA than the controlled sponge coated with THF (i.e. the Example 1 polymers performed significantly better than THF). The sponges coated with nucleobase-containing polymers (i.e. Ms22, Ms23, Ms26, Ms27, Ms28, Ms29) recovered more cfDNA than the sponge coated with non-nucleobase polymer (Ms13). In other words, nucleobase containing polymers are preferred to non-nucleobase polymer (Ms13). Nucleobase-containing polymer Ms26 is particularly preferred due to its high retention and elution of cfDNA.

In a separate experiment, MsX of Example 1 was tested for capturing cfDNA. A substrate was coated with MsX polymer. A solution of TE buffer (i.e. Tris-EDTA buffer solution from Sigma Aldrich) spiked with 200 ng mononucleosomal DNA (160 bp) was incubated for 30 min at 37° C. with 1, 2 or 3 of the MsX or THF-coated sponges. These were then washed with PBS and bound DNA was eluted using 100 mM Tris-HCl pH 8.5, 1250 mM NaCl (high salt buffer with a NaCl concentration >600 mM). The DNA was quantified by High Sensitivity dsDNA Qubit™ using an assay of fluorometric quantification of DNA. Three replicates were measured for each experiment (n=3). It is found that the amount of DNA recovered by each MsX sponge is at least comparable to that of Ms 26.

### EXAMPLE 6—HAEMOCOMPATABILITY ASSAY

It is beneficial that the polymers are haemocompatible (e.g. they cause minimal blood coagulation in use). The benefit is particularly relevant for use of the polymers as illustrated in FIG. 3. To this end, FIG. 5 indicates a qualitative coagulation assay using various polymers at different concentrations of coating solutions, where ‘+’=coagulation observable, and ‘−’=no evidence of coagulation.

FIG. 5 identifies MsX (in comparison to Ms26 and MsY) as a preferred choice due to its improved haemocompatibility. It is further noted that for other polymers, blood coagulation may be mitigated by anti-coagulant agent(s). In some applications (e.g. DNA recovery from urine samples or a 10 mL venous blood sample), haemocompatibility is less relevant.

### EXAMPLE 7—DNA RECOVERY AFTER ETHYLENE OXIDE (ETO) TREATMENT

In this example, MsX-coated polyurethane sponges were treated in different processes.

In a first process, gamma radiation was used to treat the MsX-coated polyurethane sponges. It is found that the radiation negatively affects the DNA recovery despite sterilising the sponges. In the experiment, 0 kGy (i.e. no gamma radiation), 10 kGy and 25 kGy radiation were used to treat the MsX-coated sponges respectively. 10 mL buffer spiked with 10 ng/ml DNA was incubated for 1 hour with the treated sponges. Three replica sponges were used for each incubation. After incubation, the captured DNA was washed and eluted using high-electrolyte buffer. The recovered DNA from each substrate was quantified. FIG. 6 shows a significant decrease of DNA recovery after gamma radiation (10 kGy and 25 kGy).

In a second process, MsX was coated on two 3×1 cm³ polyurethane sponges (S2 and S3) respectively. S2 sponge has a specific surface area of 18 cm²/cm³, and S3 has a specific surface area of 43 cm²/cm³.

In a first experiment, 300 mg/L ethylene oxide (EtO) was used to treat the coated sponges at 45° C. at 45% humidity for 3 hours. In a second experiment, 1000 mg/L ethylene oxide was used to treat the coated sponges at 50° C. at 75% for 7 hours. Both experiments were compliant with ISO 11135-1:2007 of EtO sterilization of healthcare products. The second experiment was an enhanced EtO treatment in comparison to the first experiment.

Some of the EtO-treated sponges from the first experiment, some of the gamma radiation treated sponges from the first process, and some untreated sponges (control) were used to recover DNA. The recovery experiment was done in a similar way as described in the first process (i.e. 10 mL buffer spiked with 10 ng/ml DNA was incubated for 1 hour with the treated sponges), except that only two replica sponges were used for each incubation. The percentages of the DNA recovery were normalized and shown in FIG. 7. The EtO-treated sponges largely preserved the DNA recovery (i.e. no significant drop of DNA recovery was observed), whilst the gamma radiation treated sponges showed a significant decrease of DNA recovery.

In a further, separate experiment, some of the EtO-treated sponges from the second experiments, and some untreated sponges (control) were incubated with 10 mL plasma spiked with 10 ng/ml DNA for 1 hour. Two replica sponges were used for each incubation. After incubation, the captured DNA was washed and eluted using high-electrolyte buffer. The recovered DNA from each sponge was quantified. Table 5 below shows the results. It is noted that this further experiment used plasma spiked with DNA, and significant improvement of DNA recovery was observed (see data in Table 5).

TABLE 5
DNA recovery for EtO treated substrates

| | MsX-S2 | MsX-S3 |
|---|---|---|
| | (EtO treated, | (EtO treated, |
| | 2^{nd} experiment) | 2^{nd} experiment) |
| DNA | 25 | 40 |
| recovery | | |
| (%) | | |
| Stdev | 7.2 | 6.3 |

The control sponges gave modest amount of DNA recovery (e.g. above 1% but below 10%). Sponges underwent enhanced EtO treatment (i.e. the second experiment) showed significant improvement of DNA recovery (e.g. >10% recovery). Further, S3 sponge having high specific surface area showed significant increase of DNA recovery in comparison to S2 sponge.

### EXAMPLE 8—DNA RECOVERY AFTER DIFFERENT ETHYLENE OXIDE (ETO) TREATMENTS

In a separate experiment, polyurethane sponges were treated with MsX, Ms26 and poly(methoxyethyl methacrylate) [pMEMA], respectively. pMEMA is a control polymer that does not contain a nucleobase.

Different EtO treatments were applied to the polymer-coated sponges. The treatment conditions are set out below.

None: This denotes to a control procedure, wherein no EtO treatment was applied.

EtO treatment 1: EtO concentration=801-810 mg/L; time=12 hours; temperature=45-53.5° C.; humidity ≥50%.

EtO treatment 2: This denotes to a treatment wherein the ‘EtO treatment 1’ was repeated once.

After EtO treatments, the coated sponges were used to recover DNA. The plasma was spiked with 500 ng DNA. The addition of spiked DNA ensures that DNA-binding observations were real and were not missed simply because DNA was not present in the used plasma sample. The coated sponges were incubated in spiked plasma, followed by washing, eluting and purification of the recovered DNA. Overall, the experimental procedure was like that of the first process, as described in Example 7. The DNA was quantified by High Sensitivity dsDNA Qubit™ using an assay of fluorometric quantification of DNA.

The percentages of the DNA recovery were normalized and are shown in Table 6.

TABLE 6
DNA recovery (%) after different EtO treatments

| | None | EtO treatment 1 | EtO treatment 2 |
|---|---|---|---|
| pMEMA | 1.0 | 0.6 | 0.4 |
| MsX | 3.8 | 35.7 | 70.6 |
| Ms26 | 6.2 | 39.2 | 68.5 |

pMEMA (not containing a nucleobase) failed to recover any meaningful amount of DNA (i.e. the DNA recovery rate was 0% or close to 0%); furthermore, this polymer did not show any changes with respect to DNA recovery after various EtO treatments.

On the other hand, both MsX and Ms26 recovered detectable amount of DNA. Both polymers showed significant improvement with respect to DNA recovery after various EtO treatments, and stronger treatment conditions resulted in higher DNA recovery.

### EXAMPLE 9—NMR ANALYSIS OF Ms26 AFTER VARIOUS ETHYLENE OXIDE (ETO) TREATMENTS

Polymer Ms26 was treated with EtO under conditions as described in Example 8.

FIG. 8 shows preliminary ¹H NMR analysis of untreated and treated Ms26. Changes of a few peaks representing certain protons were observed after EtO treatments. In the first instance (left view), a peak disappeared after EtO treatments; in the second instance (right view), a peak formed after EtO treatments, and its intensity increased along with enhancement of the EtO treatment conditions. Both changes may indicate chemical and/or compositional change(s) happened to the polymer.

What is claimed:

1. A method of preparing an apparatus configured to capture a nucleic acid from a sample, comprising the steps in sequence of: (i) providing a substrate, wherein the substrate is a nucleobase-containing polymer or is provided with said polymer; and (ii) treating the substrate and/or the polymer with ethylene oxide.

2. The method according to claim 1, wherein at step (ii), the substrate and/or the polymer is treated with ethylene oxide for a period of at least 1 hour.

3. The method according to claim 1, wherein at step (ii), the substrate and/or the polymer is treated with ethylene oxide applied from an ethylene oxide/diluent mixture in which the ethylene oxide concentration is at least 100 mg/L.

4. The method according to claim 1, wherein at step (ii), the substrate and/or the polymer is treated with ethylene oxide at a temperature of at least 15° C.

5. The method according to claim 1, wherein at step (ii), the substrate and/or the polymer is treated with ethylene oxide at a humidity of at least 30%.

6. The method according to claim 1, wherein at step (ii), the substrate and/or the polymer is treated with ethylene oxide applied from an ethylene oxide/diluent mixture in which the ethylene oxide concentration is at least 300 mg/L, and wherein the treatment is for a period of at least 2 hours, at a temperature of at least 40° C., and at a humidity of at least 30%.

7. (canceled)

8. The method according to claim 6, wherein the ethylene oxide concentration is at least 800 mg/L, and wherein the treatment is for a period of at least 7 hours, at a temperature of at least 45° C., and at a humidity of at least 50%.

9. (canceled)

10. (canceled)

11. The method according to claim 1, wherein at step (ii), the substrate and/or the polymer is treated with ethylene oxide, resulting in the substrate and/or the polymer comprising one or more ethylene oxide units.

12. (canceled)

13. (canceled)

14. The method according to claim 1, comprising providing a substrate, wherein the substrate is the nucleobase-containing polymer; treating said polymer with ethylene oxide; and providing the treated polymer to a further substrate, and wherein after providing the treated polymer to the further substrate, the method comprises treating the further substrate provided with the polymer with ethylene oxide, thereby sterilising said substrate provided with said polymer.

15. (canceled)

16. (canceled)

17. (canceled)

18. The method according to claim 1, wherein the nucleobase containing polymer comprises a backbone with at least a proportion of side chains being nucleobase side chains, and wherein the polymer further comprises a positively charged moiety at physiological pH adapted to bind the nucleic acid via electrostatic interaction, and/or the polymer is adapted to bind the nucleic acid via hydrogen bonding.

19. The method according to claim 18, wherein each of the nucleobase side chains comprises a nucleobase independently selected from adenine (A), cytosine(C), guanine (G), thymine (T), uracil(U), derivatives of ACGTU thereof and mixtures thereof.

20. The method according to claim 18, wherein the polymer comprises (meth)acrylate-based monomers with nucleobase side chains, and monomers selected from 2-methoxyethyl acrylate (MEA), 2-methoxyethyl methacrylate (MEMA), diethylamino ethylacrylate (DEAEA), diethylamino ethyl methacrylate (DEAEMA), hydroxyethyl acrylate, hydroxyethyl methacrylate, methyl methacrylate, methyl acrylate, styrene, methyl styrene, glycidyl acrylate, glycidyl methacrylate, N-vinylacetamide, 2-methyl-2-nitropropyl methacrylate, acrylic acid, methacrylic acid, 2-[[(butylamino) carbonyl]oxy]ethylacrylate, dimethylamino ethyl acrylate, dimethylamino ethyl methacrylate, mono-2-(acryloyloxy)ethyl succinate, poly(ethylene glycol) methyl ether acrylate (PEGA), poly(ethylene glycol) methyl ether methacrylate (PEGMA), and mixtures thereof.

21. (canceled)

22. The method according to claim 20, wherein the polymer comprises (meth)acrylate-based monomers with nucleobase side chains selected from thymine ethyl acrylate (ThEA), Thymine acetoxyethyl methacrylate (ThAcMA) and mixtures thereof, (meth)acrylate-based monomers with poly(ethylene glycol) side chains selected from PEGA, PEGMA and mixtures thereof, and (meth)acrylate-based monomers selected from 2-methoxyethyl (meth)acrylate, diethylamino ethyl (meth)acrylate, and mixtures thereof.

23. The method according to claim 22, wherein the polymer comprises one or more of 1-90% MEA, 1-90% MEMA, 1-50% DEAEA, 1-50% PEGA, in combination with one or more of 1-50% ThEA and 1-50% ThAcMA.

24. The method according to claim 23, wherein the polymer corresponds to one of the following structures: 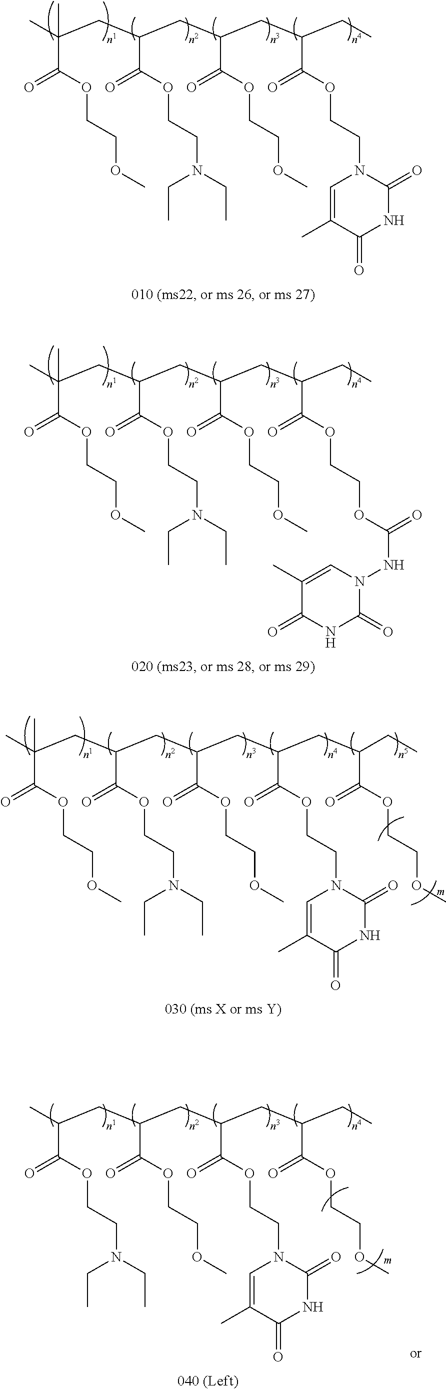 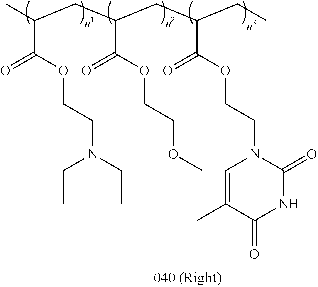
wherein the average value of m is from 1 to 50, and values of n¹-n⁵ are selected to provide the polymer with the following compositions as indicated in Table below:

| | MEMA | DEAEA | MEA | ThEA | ThAcMA | PEGA |
|---|---|---|---|---|---|---|
| | (%) | (%) | (%) | (%) | (%) | (%) |
| Ms22 | 38 | 27 | 29 | 6 | — | — |
| Ms23 | 37 | 26 | 28 | — | 9 | — |
| Ms26 | 42 | 25 | 32 | 1 | — | — |
| Ms27 | 38 | 26 | 28 | 8 | — | — |
| Ms28 | 39 | 28 | 29 | — | 4 | — |
| Ms29 | 33 | 28 | 25 | — | 14 | — |
| MsY | 39 | 30 | 29 | 1 | — | 1 |
| MsX | 47 | 15 | 36 | 1 | — | 1 |
| 040(Left) | — | 10 | 88 | 1 | — | 1 |
| 040(Right) | — | 10 | 89 | 1 | — | — |

25. An apparatus prepared according to the method of claim 1.

26. A method for preserving or improving capture of a nucleic acid from a sample, comprising the steps of: (i) providing a sample comprising a nucleic acid; (ii) providing an apparatus according to claim 25, (iii) providing the sample of step (i) to the polymer comprised in the apparatus of step (ii), and (iv) capturing the nucleic acid by the polymer.

27. The method according to claim 26, further comprising one or more of the following steps of: a. separating the nucleic acid from the sample, optionally without the use of centrifugation or pre-filtration; b. washing a polymer-nucleic acid complex to remove one or more other components from the sample; c. releasing some of or all of the nucleic acid from the polymer; d. characterising the nucleic acid; e. sequencing the nucleic acid; and f. identifying a disease state based on the characterisation of the nucleic acid.

28. The method according to claim 26, wherein the method is an extracorporeal method, or is an apheresis method.

29. (canceled)

30. (canceled)

31. (canceled)

32. (canceled)

33. (canceled)

34. A nucleobase-containing polymer, wherein the nucleobase-containing polymer is treated with ethylene oxide.

35. (canceled)
